SEQUENCE LISTING (70 sequences)

SEQ ID NO: 1 - DNA, 18 bp, artificial sequence
  Beta (27-28) 1 DNA probe
tggtgaggccctgggcag

SEQ ID NO: 2 - DNA, 18 bp, artificial sequence
  Beta (27-28) 2 DNA probe
ggtgaggcccctgggcag

SEQ ID NO: 3 - DNA, 17 bp, artificial sequence
  Beta (43) 1 DNA probe
ggttctttgagtccttt

SEQ ID NO: 4 - DNA, 17 bp, artificial sequence
  Beta (43) 2 DNA probe
ggttcttttagtccttt

SEQ ID NO: 5 - DNA, 17 bp, artificial sequence
  Beta (42 + T) 2 DNA probe
aggttcttttgagtcct

SEQ ID NO: 6 - DNA, 15 bp, artificial sequence
  IVS (2-1) 1 DNA probe
cttcagggtgagtct

SEQ ID NO: 7 - DNA, 15 bp, artificial sequence
  IVS (2-1) 2 DNA probe
cttcaggatgagtct

SEQ ID NO: 8 - DNA, 20 bp, artificial sequence
  Beta (1) 1 DNA probe
acagacaccatggtgcacct

SEQ ID NO: 9 - DNA, 20 bp, artificial sequence
  Beta (1) 2 DNA probe
acagacaccagggtgcacct

SEQ ID NO: 10 - DNA, 15 bp, artificial sequence
  Beta (8) 1 DNA probe
gaggagaagtctgcc

SEQ ID NO: 11 - DNA, 15 bp, artificial sequence
  Beta (8) 2 DNA probe
tgaggaggtctgccg

SEQ ID NO: 12 - DNA, 15 bp, artificial sequence
  Beta (8-9) 2 DNA probe
aggagaaggtctgcc

SEQ ID NO: 13 - DNA, 15 bp, artificial sequence
  Beta (37) 1 DNA probe
tacccttggacccag

SEQ ID NO: 14 - DNA, 15 bp, artificial sequence
  Beta (37) 2 DNA probe
tacccttagacccag

SEQ ID NO: 15 - DNA, 17 bp, artificial sequence
  P (+40-43) 1 DNA probe
gcaacctcaaacagaca

SEQ ID NO: 16 - DNA, 17 bp, artificial sequence
  P (+40-43) 2 DNA probe
agcaacctcagacacca

SEQ ID NO: 17 - DNA, 17 bp, artificial sequence
  P (Beta 31, IVS1) 1 DNA probe
cacccttaggctgctgg

SEQ ID NO: 18 - DNA, 17 bp, artificial sequence
  P (IVS1) 2 DNA probe
cccaccctgaggctgct

SEQ ID NO: 19 - DNA, 17 bp, artificial sequence
  Beta (31) 2 DNA probe
cccttaggtgctggtgg

SEQ ID NO: 20 - DNA, 15 bp, artificial sequence
  P (cap+1) 1 DNA probe
attgcttacatttgc

SEQ ID NO: 21 - DNA, 15 bp, artificial sequence
  P (cap+1) 2 DNA probe
attgcttccatttgc

SEQ ID NO: 22 - DNA, 15 bp, artificial sequence
  Beta (19) 1 DNA probe
aaggtgaacgtggat

SEQ ID NO: 23 - DNA, 15 bp, artificial sequence
  Beta (19) 2 DNA probe
aaggtgagcgtggat

SEQ ID NO: 24 - DNA, 15 bp, artificial sequence
  Beta (95+A) 1 DNA probe
ctgtgacaagctgca

SEQ ID NO: 25 - DNA, 15 bp, artificial sequence
  Beta (95+A) 2 DNA probe
tgtgacaaagctgca

SEQ ID NO: 26 - DNA, 15 bp, artificial sequence
  IVS (2-5) 1 DNA probe
agggtgagtctatgg

SEQ ID NO: 27 - DNA, 15 bp, artificial sequence
  IVS (2-5) 2 DNA probe
agggtgactctatgg

SEQ ID NO: 28 - DNA, 16 bp, artificial sequence
  Beta (41-42) 1 DNA probe
cagaggttctttgagt

SEQ ID NO: 29 - DNA, 16 bp, artificial sequence
  Beta (41-42) 2 DNA probe
cagaggttgagtcctt

SEQ ID NO: 30 - DNA, 15 bp, artificial sequence
  IVS (2-654) 1 DNA probe
gttaaggcaatagca

SEQ ID NO: 31 - DNA, 15 bp, artificial sequence
  IVS (2-654) 2 DNA probe
gttaaggtaatagca

SEQ ID NO: 32 - DNA, 18 bp, artificial sequence
  Beta (17) 1 DNA probe
ctgtggggcaaggtgaac

SEQ ID NO: 33 - DNA, 18 bp, artificial sequence
  Beta (17) 2 DNA probe
ctgtggggctaggtgaac

SEQ ID NO: 34 - DNA, 15 bp, artificial sequence
  Beta (71-72) 1 DNA probe
tgcctttagtgatgg

SEQ ID NO: 35 - DNA, 15 bp, artificial sequence
  Beta (71-72) 2 DNA probe
tgcctttaagtgatg

SEQ ID NO: 36 - DNA, 15 bp, artificial sequence
  Beta (71-72) 3 DNA probe
tgccttttagtgatg

SEQ ID NO: 37 - DNA, 15 bp, artificial sequence
  IVS (1-5) 1 DNA probe
caggttggtatcaag

SEQ ID NO: 38 - DNA, 15 bp, artificial sequence
  IVS (1-5) 2 DNA probe
caggttgctatcaag

SEQ ID NO: 39 - DNA, 15 bp, artificial sequence
  IVS (1-1) 1 DNA probe
tgggcaggttggtat

SEQ ID NO: 40 - DNA, 15 bp, artificial sequence
  IVS (1-1) 2 DNA probe
tgggcagtttggtat

SEQ ID NO: 41 - DNA, 15 bp, artificial sequence
  Beta (30) 2 DNA probe
ctgggcgggttggta

SEQ ID NO: 42 - DNA, 15 bp, artificial sequence
  P (-28) 1 DNA probe
gggcataagagtcag

SEQ ID NO: 43 - DNA, 15 bp, artificial sequence
  P (-28) 2 DNA probe
gggcataggagtcag

SEQ ID NO: 44 - DNA, 15 bp, artificial sequence
  P (-29) 2 DNA probe
tgggcatggaagtca

SEQ ID NO: 45 - DNA, 15 bp, artificial sequence
  P (-30) 1 DNA probe
ctgggcataaaagtc

SEQ ID NO: 46 - DNA, 15 bp, artificial sequence
  P (-30) 2 DNA probe
ctgggcacaaaagtc

SEQ ID NO: 47 - DNA, 15 bp, artificial sequence
  P (-31) 2 DNA probe
gctgggcgtaaaagt

SEQ ID NO: 48 - DNA, 15 bp, artificial sequence
  P (-32) 2 DNA probe
ggctgggaataaaag

SEQ ID NO: 49 - DNA, 20 bp, artificial sequence
  Beta (14-15) 1 DNA probe
tactgccctgtggggcaagg

SEQ ID NO: 50 - DNA, 20 bp, artificial sequence
  Beta (14-15) 2 DNA probe
tactgccctggtggggcaag

SEQ ID NO: 51 - DNA, 15 bp, artificial sequence
  HbE (26) 1 DNA probe
tggtggtgaggccct

SEQ ID NO: 52 - DNA, 15 bp, artificial sequence
  HbE (26) 2 DNA probe
tggtggtaaggccct

SEQ ID NO: 53 - DNA, 15 bp, artificial sequence
  P(cs)1 DNA probe
ataccgttaagctgg

SEQ ID NO: 54 - DNA, 15 bp, artificial sequence
  P(cs)2 DNA probe
ataccgtcaagctgg

SEQ ID NO: 55 - DNA, 15 bp, artificial sequence
  P(qs)1 DNA probe
gcctccctggacaag

SEQ ID NO: 56 - DNA, 15 bp, artificial sequence
  P(qs)2 DNA probe
gcctccccggacaag

SEQ ID NO: 57 - DNA, 15 bp, artificial sequence
  P(hbs)1 DNA probe
actcctgaggagaag

SEQ ID NO: 58 - DNA, 15 bp, artificial sequence
  P(hbs) 2 DNA probe
actcctgtggagaag

SEQ ID NO: 59 - DNA, 15 bp, artificial sequence
  P (duan) 1 DNA probe
gtggacgacatgccc

SEQ ID NO: 60 - DNA, 15 bp, artificial sequence
  P (duan) 2 DNA probe
gtggacgccatgccc

SEQ ID NO: 61 - DNA, 15 bp, artificial sequence
  P (hbm) 1 DNA probe
taagggccacggcaa

SEQ ID NO: 62 - DNA, 15 bp, artificial sequence
  P (hbm) 2 DNA probe
taagggctacggcaa

SEQ ID NO: 63 - DNA, 15 bp, artificial sequence
  P (hbm) 3 DNA probe
cgacctgcacgcgca

SEQ ID NO: 64 - DNA, 15 bp, artificial sequence
  P (hbm) 4 DNA probe
cgacctgtacgcgca

SEQ ID NO: 65 - DNA, 15 bp, artificial sequence
  P (hbm) 5 DNA probe
aagaaagtgctcggt

SEQ ID NO: 66 - DNA, 15 bp, artificial sequence
  P (hbm) 6 DNA probe
aagaaagagctcggt

SEQ ID NO: 67 - DNA, 15 bp, artificial sequence
  P (hbm) 7 DNA probe
tgagctgcactgtga

SEQ ID NO: 68 - DNA, 15 bp, artificial sequence
  P (hbm) 8 DNA probe
tgagctgtactgtga

SEQ ID NO: 69 - DNA, 15 bp, artificial sequence
  P (hbm) 9 DNA probe
gaaggctcatggcaa

SEQ ID NO: 70 - DNA, 15 bp, Artificial sequence
  P (hbm) 10 DNA probe
gaaggcttatggcaa

(19) United States
(12) Patent Application Publication
(10) Pub. No.: US 2003/0186230 A1
(43) Pub. Date: Oct. 2, 2003

(54) Medium and low density gene chips

(76) Inventor: Ye Bangce, Shanghai (CN)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(21) Appl. No.: 09/912,673

(22) Filed: Jul. 23, 2001

(51) Int. Cl. C12Q 1/68; C07H 21/04; C12M 1/34

(52) U.S. Cl. 435/6; 435/287.2; 536/23.2; 536/24.3

(57) ABSTRACT

This invention makes public a kind of DNA chip for diagnosing the mutation of the hereditary anemia related genes with the character of fixed specific NA probes for testing the mutation of hereditary anemia related gent on the glass slide, silica plate, membrane and macromolecular materials. In comparison with current techniques, in this invention a 70×4 DNA probe is fixed on the surface of a carrier the size of a microscope slide, and this probe can detect hereditary anemia such as α-, or β-thalassemia, and hemoglobin abnormality caused by related gent mutation. The invention has the statistic characteristics of parallel analysis and multiple analysis. Under the specific elution conditions, the completely matched and single-base-non-matched hybridization can be distinguished. Consequently, this DNA chip is appropriate for early diagnosis and prenatal screening of hereditary anemia.

### BACKGROUND OF THE INVENTION

This invention is a kind of DNA chip for diagnosing the mutation of the hereditary anemia related genes.

China is a big country where a large part of the population is afflicted with the hereditary disease. The hereditary disease is a kind of disease caused by the modification of the genetic materials in human germ cells or fertilized ova and transferred from parental generation to progeny, and usually include 3 big groups: monogenic hereditary disease, chromosome hereditary disease and polygenic hereditary disease. Considering the whole world, at least 2% of newborns are afflicted with evident congenital abnormality. Though the incidence of most hereditary diseases (except polygenic hereditary disease) is low, there are a great variety of hereditary diseases. There are more than 4000 monogenic hereditary diseases, more than 100 chromosome hereditary diseases, and no less than 100 polygenic diseases. Moreover, on the average more than 100 new monogenic hereditary diseases have been being found per years in the world, which reflects the severity of the problems hereditary diseases have brought to medicine, society and families.

For most of the hereditary diseases, there is no effective treatment at present, and hence the prevention seems particularly important. If fetuses can be diagnosed to determine whether they are afflicted with hereditary diseases, artificial abortion can be conducted on ill fetuses to prevent their coming into the world. Prenatal genetic screening supplemented with artificial abortion has been approved to be an effective method for preventing hereditary diseases that seriously harm health. Presently there are about 50,000,000 disabled persons in our country, a considerable number of which are caused by hereditary diseases. Because the rate of consanguineous marriage is high in remote regions and some rural areas, the incidence of hereditary diseases still tends to increase. Therefore, widely implementing prenatal genetic screening to eliminate the birth of congenitally defected infants and lower disease incidence plays an important role in ensuring eugenically superior birth and rearing, and improving population quality.

Because the genes as well as the mechanism of gene mutation of a great deal of hereditary diseases, particularly the monogenic Mendelian hereditary disease has been clarified through research, we can precisely diagnose most of the hereditary diseases at the gene level. The genetic screening of newborns is very common in many advanced countries. For instance, the US law has stipulated that the screening for phenylketonuria must be conducted on all the newborns as early as 1960s. As a result the appearance of newborns with disturbance of intelligence caused by phenylketonuria has been completely eliminated there. The reason is that if phenylketonuria infants can be treated with special recipe therapy immediately after birth, the disease symptoms will not appear. Besides, the genetic screening of specific newborns, such as that of black infants for sickle cell anemia, or that of Jewish infants for ______, has been implemented prevalently. Nevertheless in China, because there are a great variety of hereditary diseases and the traditional genetic screening method needs relatively expensive instruments, professional personnel and accordingly high expenses, our country has not done adequately on the popularization of prenatal and postpartum genetic screening, which will pose a profound impact on such issues as sustainable development, improvement of population quality and burden of social security. For instance, in the case of phenylketonuria, a disease of autosomal recessive inheritance, the patient cannot transform phenylketonuria into Tyrosine through normal metabolic pathway due to severe deficiency of phenylketonuria hydroxylase, and hence the metabolite of phenylketonuria is accumulated in a large amount in the body, impairing the patient's nerve system and causing disturbance of intelligence. The incidence of this disease is 1/10000 in the regions of Europe and America, and is 16000 in China. The most successful case for controlling the incidence of hereditary diseases by the combination of prenatal genetic screening and artificial abortion is the control of the β-thalassemia in the Mediterranean region. Thalassemia is a hereditary hemolytic anemia, the most common monogenic hereditary disease with the highest incidence in the world. It has relative high incidence in the Mediterranean region, Southeast Asia, India Sub-continent and the south of China (provinces such as Guangdong, Guangxi, Hainai, Guizhou and Sichuang), as well as among Afro-Americas. The world Health Organization predicted in 1985 that there would be about 7% of the world population carrying the genetic gene of hemoglobinopathy by the end of this century. According to statistic data, the incidence of α-thalassemia and β-thalassemia is 5.15% and 3% respectively in Guangdong Province, 4.8% and 16.8-24.9% respectively in Hainan Province, 3% and 6% respectively in Hong Kong, 6.2% and 3.4% respectively in Macao, and the incidence of a-thalassemia is 14.9% in the Nanning region (in a specific area it even reaches 26%), Guangxi Zhuang Autonomous Region etc. As there has emerged no specific treatment for thalassemia at present, the basic measure is prenatal diagnosis to eliminate the birth of congenitally ill infants and reach the objective of prevention and eugenically superior birth. Since 1975, the Mediterranean countries, Cyprus, Italia, Sardinia and Greece have successfully implemented the method of the prenatal genetic screening and selective artificial abortion, and hence the incidence of thalassemia there has significantly been lowered by more than 5 times.

Because there are a great variety of hereditary diseases, there are plenty of mutations of the related gene for every hereditary disease. The traditional diagnosing methods for hereditary diseases are time consuming and energy consuming as well as expensive, not suitable for application to the nationwide prenatal genetic screening in China. With modem techniques, hereditary diseases such as aα, or β-thalassemia, sickle cell anemia, hemoglobin abnormality and glucose 6-phosphate dehydrogenase deficiency have been mainly diagnosed through testing on the mutation of related diseases and analysis of the enzymatic activity. The tests for disease-related gene mutation are mainly as follows:

PCR-RFLP (restriction fragment length polymorphism): In this method that can only be applied to the case that mutation alters a certain cleavage site, the gene amplification materials were digested with many kinds of restriction endonucleases, and different gene sequences will generate relevant different electophoretic patterns (Mercier, B. et al., Eur. J. Immunogenetics, 21, 105, 1994). An improvement of this method is to apply PCR-AIRS (artificial introduction of restriction sites) to the mutated site. (Cotton, R. G. H. Mut. Res., 285, 125, 1993).

PCR-SSO (sequence specific oligonucleotide) or PCR-ASO (allele specific oligonucleotide (Cotton, R. G. H. Mut. Res., 285, 125, 1993; Saiki, R. K. et al., Nature, 324, 163, 1986): the to-be-tested gene is amplified through PCR and then is hybridized with the 15-20 bp-labeled wild-type probe and mutation-type probe respectively. In this method, it is necessary to conduct the isotape labeling or the labeling with digoxin, biotin, peroxidase etc., so the analyzing process is complicated. Therefore, this method is not suitable for quick analysis.

PCR-SSP (sequence specific primers) or ASPCR (allele specific PCR): The principle is to design a non-matched based in the primer according to the property of the known mutated site, so that only the mutation-type gene or wild-type gene can be amplified. This method is relative quick, simple and convenient. (Wu, D. Y., et al., Proc. Natl. Acad. Sci. U.S.A., 86, 2757, 1986; rust, S., et al., Nucleic Acids Research, 21, 3623, 1993; Newton, R. et al., Nucleic Acids Research, 17, 2503, 1989).

PCR-SSCP (single-stranded conformation polymorphism): SSCP denotes the property of single DNA that the electophoretic migration rate of the single DNA in the neutral page changes with the relevant conformation, which can be used for testing the gene differentiation. The earliest application of this method is to the researches on the mutation of cancer gene sites and the human genomic polymorphism (Orita, M., et al., Proc. Natl. Acad. Sci. U.S.A., 86, 2766, 1989). This method can be applied to testing the existence of the gene differentiation exclusively, but not to determining the site and contents of the mutation. DNA-Sequence-Assaying Method: This is the most direct viewing and precise method, but the relevant techniques are complicated and operating expenses are high, so it cannot be taken as a routine method.

### BRIEF SUMMARY OF THE INVENTION

In summary, in the existing techniques for diagnosing relevant disease-related gene mutation, there exist the problems of complicated operation, long operating time needed, high cost and difficulty for the realization of automation and mass-sample parallel analysis etc.

The purpose of this invention is to provide a DNA chip for simultaneously testing all hereditary anemia related gene mutations for diagnosis.

### DETAILED DESCRIPTION OF THE INVENTION

In order to reach the said purpose, measures are taken as follows:

The DNA chip for diagnosing the hereditary anemia related gene mutations is a specific DNA probe fixed on the glass slide, silica plate, membrane and macromolecular materials for testing the said mutations. The varieties of the said probe are as follows:

α-thalassemia

| | |
|---|---|
| β (27-28) 1 | |
| TGG TGA GGC CCT GGG CAG | (SEQ ID NO:1) |
| β (27-28) 2 | |
| GGT GAG GCC CCT GGG CAG | (SEQ ID NO:2) |
| β (43) 1 | |
| GGT TCT TTG AGT CCT TT | (SEQ ID NO:3) |
| β (43) 2 | |
| GGT TCT TTT AGT CCT TT | (SEQ ID NO:4) |
| β (42 + T) 2 | |
| AGG TTC TTT TGA GTC CT | (SEQ ID NO:5) |
| IVS (2-1) 1 | |
| CTT CAG GGT GAG TCT | (SEQ ID NO:6) |
| IVS (2-1) 2 | |
| CTT CAG GAT GAG TCT | (SEQ ID NO:7) |
| β (1) 1 | |
| ACA GAC ACC ATG GTG CAC CT | (SEQ ID NO:8) |
| β (1) 2 | |
| ACA GAC ACC AGG GTG CAC CT | (SEQ ID NO:9) |
| β (8) 1 | |
| GAG GAG AAG TCT GCC | (SEQ ID NO:10) |
| β (8) 2 | |
| TGA GGA GGT CTG CCG | (SEQ ID NO:11) |
| β (8-9) 2 | |
| AGG AGA AGG TCT GCC | (SEQ ID NO:12) |
| β (37) 1 | |
| TAC CCT TGG ACC CAG | (SEQ ID NO:13) |
| β (37) 2 | |
| TAC CCT TAG ACC CAG | (SEQ ID NO:14) |
| P (+ 40-43) 1 | |
| GCA ACC TCA AAC AGA CA | (SEQ ID NO:15) |
| P (+ 40-43) 2 | |
| AGC AAC CTC AGA CAC CA | (SEQ ID NO:16) |
| P (β 31,IVS1) 1 | |
| CAC CCT TAG GCT GCT GG | (SEQ ID NO:17) |
| P (IVS1) 2 | |
| CCC ACC CTG AGG CTG CT | (SEQ ID NO:18) |
| β (31) 2 | |
| CCC TTA GGT GCT GGT GG | (SEQ ID NO:19) |
| P (cap + 1) 1 | |
| ATT GCT TAC ATT TGC | (SEQ ID NO:20) |
| P (cap + 1) 2 | |
| ATT GCT TCC ATT TGC | (SEQ ID NO:21) |
| β (19) 1 | |
| AAG GTG AAC GTG GAT | (SEQ ID NO:22) |
| β (19) 2 | |
| AAG GTG AGC GTG GAT | (SEQ ID NO:23) |
| β (95 + A) 1 | |
| CTG TGA CAA GCT GCA | (SEQ ID NO:24) |
| β (95 + A) 2 | |
| TGT GAC AAA GCT GCA | (SEQ ID NO:25) |
| IVS (2-5) 1 | |
| AGG GTG AGT CTA TGG | (SEQ ID NO:26) |
| IVS (2-5) 2 | |
| AGG GTG ACT CTA TGG | (SEQ ID NO:27) |
| β (41-42) 1 | |
| CAG AGG TTC TTT GAG T | (SEQ ID NO:28) |
| β (41-42) 2 | |
| CAG AGG TTG AGT CCT T | (SEQ ID NO:29) |
| IVS (2-654) 1 | |
| GTT AAG GCA ATA GCA | (SEQ ID NO:30) |
| IVS (2-654) 2 | |
| GTT AAG GTA ATA GCA | (SEQ ID NO:31) |
| β (17) 1 | |
| CTG TGG GGC AAG GIG AAC | (SEQ ID NO:32) |
| β (17) 2 | |
| CTG TGG GGC TAG GTG AAC | (SEQ ID NO:33) |
| β (71-72) 1 | |
| TGC CTT TAG TGA TGG | (SEQ ID NO:34) |
| β (71-72) 2 | |
| TGC CTT TAA GTG ATG | (SEQ ID NO:35) |
| β (71-72) 3 | |
| TGC CTT TTA GTG ATG | (SEQ ID NO:36) |
| IVS (1-5) 1 | |
| CAG GTT GGT ATC AAG | (SEQ ID NO:37) |
| IVS (1-5) 2 | |
| CAG GTT GCT ATC AAG | (SEQ ID NO:38) |
| IVS (1-1) 1 | |
| TGG GCA GGT TGG TAT | (SEQ ID NO:39) |
| IVS (1-1) 2 | |
| TGG GCA GTT TGG TAT | (SEQ ID NO:40) |
| β (30) 2 | |
| CTG GGC GGG TTG GTA | (SEQ ID NO:41) |
| P (−28) 1 | |
| GGG CAT AAG AGT CAG | (SEQ ID NO:42) |
| P (−28) 2 | |
| GGG CAT AGG AGT CAG | (SEQ ID NO:43) |
| P (−29) 2 | |
| TGG GCA TGG AAG TCA | (SEQ ID NO:44) |
| P (−30) 1 | |
| CTG GGC ATA AAA GTC | (SEQ ID NO:45) |
| P (−30) 2 | |
| CTG GGC ACA AAA GTC | (SEQ ID NO:46) |
| P (−31) 2 | |
| GCT GGG CGT AAA AGT | (SEQ ID NO:47) |
| P (−32) 2 | |
| GGC TGG GAA TAA AAG | (SEQ ID NO:48) |
| β (14-15) 1 | |
| TAC TGC CCT GTG GGG CAA GG | (SEQ ID NO:49) |
| β (14-15) 2 | |
| TAC TGC CCT GGT GGG GCA AG | (SEQ ID NO:50) |
| HbE (26) 1 | |
| TGG TGG TGA GGC CCT | (SEQ ID NO:51) |
| HbE (26) 2 | |
| TGG TGG TAA GGC CCT | (SEQ ID NO:52) |

α-thalassemia, HbH, HbS and HbM hemoglobin abnormality

Constant Spring Mutation

| | | |
|---|---|---|
| P (cs) 1 | ATA CCG TTA AGG TGG | (SEQ ID NO:53) |
| P (cs) 2 | ATA CCG TCA AGC TGG | (SEQ ID NO:54) |

Quong SZE Mutation

| | | |
|---|---|---|
| P (qs) 1 | GCC TCC CTG GAC AAG | (SEQ ID NO:55) |
| P (qs) 2 | GCC TCC CCG GAC AAG | (SEQ ID NO:56) |

HbS Sickle Cell Anemia Mutation

| | | |
|---|---|---|
| P (hbs) 1 | ACT CCT GAG GAG AAG | (SEQ ID NO:57) |
| P (hbs) 2 | ACT CCT GTG GAG AAG | (SEQ ID NO:58) |

Duan Mutation

| | | |
|---|---|---|
| P (duan) 1 | GTG GAC GAC ATG CCC | (SEQ ID NO:59) |
| P (duan) 2 | GTG GAC GCC ATG CCC | (SEQ ID NO:60) |

HbM Mutation

| | | |
|---|---|---|
| P (hbm) 1 | TAA GGG CCA CGG CAA | (SEQ ID NO:61) |
| P (hbm) 2 | TAA GGG CTA CGG CAA | (SEQ ID NO:62) |
| P (hbm) 3 | CGA CCT GCA CGC GCA | (SEQ ID NO:63) |
| P (hbm) 4 | CGA CCT GTA CGC GCA | (SEQ ID NO:64) |
| P (hbm) 5 | AAG AAA GTG CTC GGT | (SEQ ID NO:65) |
| P (hbm) 6 | AAG AAA GAG CTC GGT | (SEQ ID NO:66) |
| P (hbm) 7 | TGA GCT GCA CTG TGA | (SEQ ID NO:67) |
| P (hbm) 8 | TGA GCT GTA CTG TGA | (SEQ ID NO:68) |
| P (hbm) 9 | GAA GGC TCA TGG CAA | (SEQ ID NO:69) |
| P (hbm) 10 | GAA GGC TTA TGG CAA | (SEQ ID NO:70) |

In comparison with current techniques, in this invention a 70×4 DNA probe is fixed on the surface of a carrier the size of a microscope slide, and this probe can detect hereditary anemia such as α-, or β-thalassemia, and hemoglobin abnormality caused by the related mutation of genes. The relevant statistic analyses have the characteristics of parallel analysis and multiple analysis. Under the specific elution conditions, the completely matched and single-base-non-matched hybridization can be distinguished. Consequently, this DNA chip is appropriate for early diagnosis and prenatal screening of hereditary anemia. The sole chip can test all the gene mutation sites causing the hereditary anemia of Chinese ethnic groups. For β-thalassemia, the mutated sites include β41-42(-TCTT), IVS2-654(C-T), β17(A-T), -28(A-G), P β71-72(+A or +T), IVS1-5(G-C), -30(T-C), β14-15(+G), IVS1-1(G-C), HbE26(G-A), β27-28(+C), -29(A-G), β43(G-T), IVS2-1(G-T), β1(T-G), IVS(2-5), β(30), -31, -32, β(31), cap+1, β(19), β(95+A), β(42+T), β(1), β(8-9), +40-43, β(37), β(31) mutation; for hemoglobin abnormality, the mutated sites include Constant Spring (T-C), Quong SZE (T-C), HbS(β36 A-T), Duan (α75 A-C), HbM (α58 C-T, α87 C-T, β68 T-A, β93 C-T, β64 C-T) mutation. The developed gene chip for diagnosing common Chinese hereditary diseases will be the best means for prenatal hereditary screening. Several mutation-testing probes for the genes of several most common hereditary anemia diseases that have the highest incidence in China, such as thalassemia and hemoglobin abnormality, have been designed and fixed on the chip through the automatic micro-controlling technique according to a certain array sequence. Thus the gene chip for diagnosing the most common hereditary anemia has been prepared. If the comprehensive prenatal hereditary screening has been conducted with this technique, combined with selective artificial abortion, the cases of hereditary anemia that severely impaired human health will be eliminated, which is of momentous significance for improving the population quality and alleviating the burden of society and families.

### DESCRIPTION OF THE DRAWINGS

This invention is illustrated in detail with the combination of attached figures and samples as follows:

FIG. 1: DNA specific pattern for diagnosing hereditary anemia.

FIG. 2: Resulting pattern from testing mutated β14-15 (+G) of thalassemia patients.

FIG. 3: Hybridization result from testing mutated β14-15 (+G) of thalassemia patients.

FIG. 4: Hybridization result from testing mutated HbS sickle cell anemia patient.

The purpose of this invention is thus realized: The designed specific probe is fixed on the surface of a kind of carrier such as glass slide, silica plate, membrane and macromolecular materials, and the method of surface-arrayed specific DNA probes is applied to testing a variety of gene mutations of hereditary diseases.

The most common Chinese gene mutations among the hereditary anemia related gene mutations are selected as follows: For P-thalassemia, the mutated sites include β41-42(-TCTT), IVS2-654 (C-T), β17(A-T), -28(A-G), β71-72(+A or +T), IVS1-5(G-C), -30(T-C), β14-15(+G), IVS1-1(G-C), HbE26(G-A), β27-28(+C), -29(A-G), β43(G-T), IVS2-1(G-T), β1(T-G), IVS (2-5), β(30), -31, -32, β(31), cap+1β(19), β(95+A), βR2+T), β(1), β(8), β(8-9), +40-43, β(37), (31) mutation; for hemoglobin abnormality, the mutated sites include Constant Spring (T-C), Quong SZE(T-C), HbS(β6 A-T), Duan (α75 A-C), HbM (α58 C-T, α87 C-T, β68 T-A, β93 C-T, β64 C-T) mutation; The said mutated genes are used to synthesize relevant wild-type and mutation-type DNA probes, and they are fixed on the surface of the chips. The related probes are as follows:

β-thalassemia

| | |
|---|---|
| β (27-28) 1 | |
| TGG TGA GGC CCT GGG CAG | (SEQ ID NO:1) |
| β (27-28) 2 | |
| GGT GAG GCC CCT GGG CAG | (SEQ ID NO:2) |
| β (43) 1 | |
| GGT TCT TTG AGT CCT TT | (SEQ ID NO:3) |
| β (43) 2 | |
| GGT TCT TTT AGT CCT TT | (SEQ ID NO:4) |
| β (42 + T) 2 | |
| AGG TTC TTT TGA GTC CT | (SEQ ID NO:5) |
| IVS (2-1) 1 | |
| CTT CAG GGT GAG TCT | (SEQ ID NO:6) |
| IVS (2-1) 2 | |
| CTT CAG GAT GAG TCT | (SEQ ID NO:7) |
| β (1) 1 | |
| ACA GAC ACC ATG GTG CAC CT | (SEQ ID NO:8) |
| β (1) 2 | |
| ACA GAC ACC AGG GTG CAC CT | (SEQ ID NO:9) |
| β (8) 1 | |
| GAG GAG AAG TCT GCC | (SEQ ID NO:10) |
| β (8) 2 | |
| TGA GGA GGT CTG CCG | (SEQ ID NO:11) |
| β (8-9) 2 | |
| AGG AGA AGG TCT GCC | (SEQ ID NO:12) |
| β (37) 1 | |
| TAC CCT TGG ACC CAG | (SEQ ID NO:13) |
| β (37) 2 | |
| TAC CCT TAG ACC CAG | (SEQ ID NO:14) |
| P (+40-43) 1 | |
| GCA ACC TCA AAC AGA CA | (SEQ ID NO:15) |
| P (+40-43) 2 | |
| AGC AAC CTC AGA CAC CA | (SEQ ID NO:16) |
| P (β31, IVS1) 1 | |
| CAC CCT TAG GCT GCT GG | (SEQ ID NO:17) |
| P (IVS1) 2 | |
| CCC ACC CTG AGG CTG CT | (SEQ ID NO:18) |
| β (31) 2 | |
| CCC TTA GGT GCT GGT GG | (SEQ ID NO:19) |
| P (cap + 1) 1 | |
| ATT GCT TAC ATT TGC | (SEQ ID NO:20) |
| P (cap + 1) 2 | |
| ATT GCT TCC ATT TGC | (SEQ ID NO:21) |
| β (19) 1 | |
| AAG GTG AAC GTG GAT | (SEQ ID NO:22) |
| β (19) 2 | |
| AAG GTG AGC GTG GAT | (SEQ ID NO:23) |
| β (95 + A) 1 | |
| CTG TGA CAA GCT GCA | (SEQ ID NO:24) |
| β (95 + A) 2 | |
| TGT GAC AAA GCT GCA | (SEQ ID NO:25) |
| IVS (2-5) 1 | |
| AGG GTG AGT CTA TGG | (SEQ ID NO:26) |
| IVS (2-5) 2 | |
| AGG GTG ACT CTA TGG | (SEQ ID NO:27) |
| β (41-42) 1 | |
| CAG AGG TTC TTT GAG T | (SEQ ID NO:28) |
| β (41-42)2 | |
| CAG AGG TTG AGT CCT T | (SEQ ID NO:29) |
| IVS (2-654) 1 | |
| GTT AAG GCA ATA GCA | (SEQ ID NO:30) |
| IVS (2-654)2 | |
| GTT AAG GTA ATA GCA | (SEQ ID NO:31) |
| β (17) 1 | |
| CTG TGG GGC AAG GTG AAC | (SEQ ID NO:32) |
| β (17) 2 | |
| CTG TGG GGC TAG GTG AAC | (SEQ ID NO:33) |
| β (71-72) 1 | |
| TGC CTT TAG TGA TGG | (SEQ ID NO:34) |
| β (71-72) 2 | |
| TGC CTT TAA GTG ATG | (SEQ ID NO:35) |
| β (71-72) 3 | |
| TGC CTT TTA GTG ATG | (SEQ ID NO:36) |
| IVS (1-5) 1 | |
| CAG GTT GGT ATC AAG | (SEQ ID NO:37) |
| IVS (1-5)2 | |
| CAG GTT GCT ATC AAG | (SEQ ID NO:38) |
| IVS (1-1) 1 | |
| TGG GCA GGT TGG TAT | (SEQ ID NO:39) |
| IVS (1-1) 2 | |
| TGG GCA GTT TGG TAT | (SEQ ID NO:40) |
| β (30) 2 | |
| CTG GGC GGG TTG GTA | (SEQ ID NO:41) |
| P (−28) 1 | |
| GGG CAT AAG AGT CAG | (SEQ ID NO:42) |
| P (−28) 2 | |
| GGG CAT AGG AGT CAG | (SEQ ID NO:43) |
| P (−29) 2 | |
| TGG GCA TGG AAG TCA | (SEQ ID NO:44) |
| P (−30) 1 | |
| CTG GGC ATA AAA GTC | (SEQ ID NO:45) |
| P (−30) 2 | |
| CTG GGC ACA AAA GTC | (SEQ ID NO:46) |
| P (−31) 2 | |
| GCT GGG CGT AAA AGT | (SEQ ID NO:47) |
| P (−32) 2 | |
| GGC TGG GAA TAA AAG | (SEQ ID NO:48) |
| β (14-15) 1 | |
| TAC TGC CCT GTG GGG CAA GG | (SEQ ID NO:49) |
| β (14-15) 2 | |
| TAC TGC CCT GGT GGG GCA AG | (SEQ ID NO:50) |
| HbE (26) 1 | |
| TGG TGG TGA GGC CCT | (SEQ ID NO:51) |
| HbE (26) 2 | |
| TGG TGG TAA GGC CCT | (SEQ ID NO:52) |

α-thalassemia, HbH, HbS and HbM hemoglobin abnormality.

Constant Spring Mutation

| | | |
|---|---|---|
| P (cs) 1 | ATA CCG TTA AGG TGG | (SEQ ID NO:53) |
| P (cs) 2 | ATA CCG TCA AGC TGG | (SEQ ID NO:54) |

Quong SZE Mutation

| | | |
|---|---|---|
| P (qs) 1 | GCC TCC CTG GAC AAG | (SEQ ID NO:55) |
| P (qs) 2 | GCC TCC CCG GAC AAG | (SEQ ID NO:56) |

HbS Sickle Cell Anemia Mutation

| | | |
|---|---|---|
| P (hbs) 1 | ACT CCT GAG GAG AAG | (SEQ ID NO:57) |
| P (hbs) 2 | ACT CCT GTG GAG AAG | (SEQ ID NO:58) |

Duan Mutation

| | | |
|---|---|---|
| P (duan) 1 | GTG GAC GAC ATG CCC | (SEQ ID NO:59) |
| P (duan) 2 | GTG GAC GCC ATG CCC | (SEQ ID NO:60) |

HbM Mutation

| | | |
|---|---|---|
| P (hbm) 1 | TAA GGG CCA CGG CAA | (SEQ ID NO:61) |
| P (hbm) 2 | TAA GGG CTA CGG CAA | (SEQ ID NO:62) |
| P (hbm) 3 | CGA CCT GCA CGC GCA | (SEQ ID NO:63) |
| P (hbm) 4 | CGA CCT GTA CGC GCA | (SEQ ID NO:64) |
| P (hbm) 5 | AAG AAA GTG CTC GGT | (SEQ ID NO:65) |
| P (hbm) 6 | AAG AAA GAG CTC GGT | (SEQ ID NO:66) |
| P (hbm) 7 | TGA GCT GCA CTG TGA | (SEQ ID NO:67) |
| P (hbm) 8 | TGA GCT GCA CTG TGA | (SEQ ID NO:68) |
| P (hbm) 9 | GAA GGC TCA TGG CAA | (SEQ ID NO:69) |
| P (hbm) 10 | GAA GGC TTA TGG CAA | (SEQ ID NO:70) |

Preparation of diagnosing chip for hereditary anemia: Synthesize the above-listed DNA probes, and modify the 5′ termini with the amino-group or other active groups; Fix each kind of the probes repeatedly for 3 times onto the active surface of the carrier to prepare the specific DNA chip for diagnosing hereditary anemia.

Processing of sample: Amplify the target gene or mutated area of the to-be-tested sample with the designed PCR primer; label the 5′ terminus of PCR primer with fluorescence or add the fluorescence labeled dNTP during the process of PCR amplification.

Hybridizing process on the surface of DNA chip: Hybridize the fluorescence-labeled PCR amplification product with the DNA probe on the surface of the hereditary-anemia-diagnosing chip and distinguish between the completely matched hybridization and the single-base-non-matched hybridization under the specific elution condition.

Testing for hybridizing signal: After the hybridization, test the hybridizing signal on the DNA chip with confocal fluorescent microscope or fluorescent scanner for to-be-tested gene mutations.

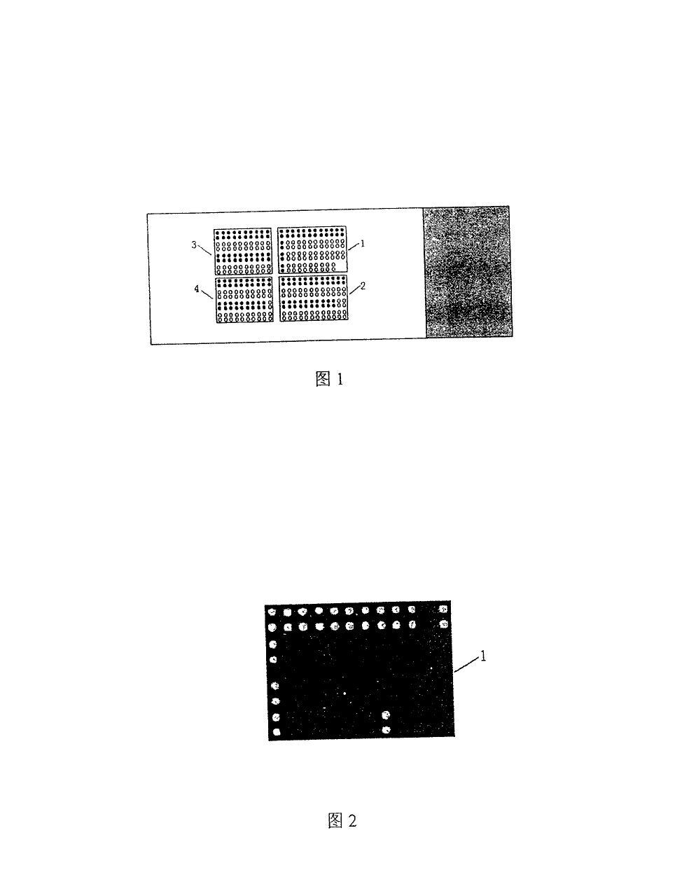

FIG. 1 shows the DNA specific pattern for diagnosing hereditary anemia. 1: Glass slide; 2:20×30 mutation sequence probes, sequentially from left to right: IVS1-5(G-C), IVS1-1(G-A), HbE26(G-A), β27-28 (+C), β17 (A-T), β14-15 (+G), -28(A-G, -29(A-G), -30(T-C), -31, -32, β31(T-G), CAP+1, β(19), β(8), β(8-9), +40-43, β(30), β(31), β(37); 3:20×3 normal sequence probes corresponding with the mutation probes in the above-listed “2”; 4:20×3 mutation sequence probes, sequentially from left to right: β71-72 (+A OR +T), β41-42 (-TCTT), β(42+T), β43(G-T), IVS2-654(C-T), IVS2-1(G-T), IVS (2-5), β(95+A); Constant Spring Mutation, Quong SZE mutation, HbS sickle cell anemia mutation, Duan mutation, HbM mutation of No. 1, 2, 3, 4, 5, β41-42(-TCTT), IVS2-654(C-T); 5:20×3 normal sequence probes corresponding with the mutation probes in the above-listed “4”.

FIG. 2 shows the resulting pattern from testing mutated β14-15 (+G) of thalassemia patients. 6:β14-15 (G) mutated site signal.

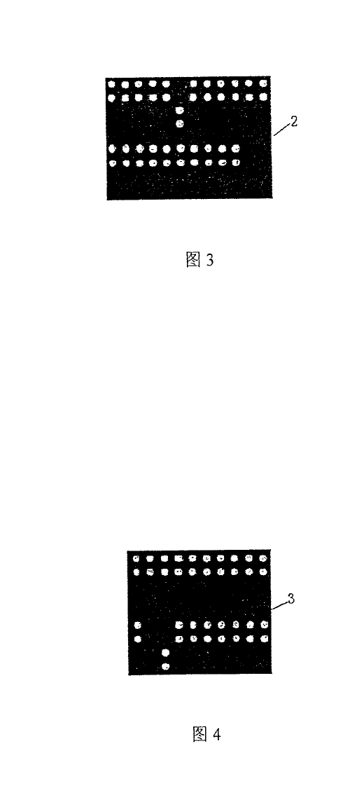

FIG. 3 shows hybridization result from testing mutated β14-15 (+G) of thalassemia patents. 7 and 8: Normal sequence signals; 6:β14-15 (+G) mutated site signal.

FIG. 4: Hybridizing result from testing mutated HbS sickle cell anemia patient. 7 and 8: Normal sequence signals; 9: HbS mutated site signal for sickle cell anemia.

### EXAMPLE 1

Use the chip of this invention to test thalassemia patients with β14-15 (+G) mutation. Take a blood sample of 1 ml from the patient to be tested and extract the genomic DNA of the patient with the commercialized genomic DNA extraction kit or the standard phenol extracting/ethanol depositing method. Amplify the exon 1, 2, 3 of β globin gene with the designed PCR (polymerase chain reaction) primers of three groups (5′ terminus of one primer modified with fluorescence). The amplified volume is 50 μl. Mix the PCR product and add 3 M pH 5.2 acidic acid buffer with the volume 1/10 time that of the sample. Add −20 C. refrigerated 100% ethanol with the volume 2.5 times that of the sample. Evenly mix the sample and let it stand at 20 C. for 30 min. Centrifuge at 1300 rpm for 10 min. and wash with 70% ethanol. Then allow to deposit and dry.

Hybridizing process on the surface of DNA chip:

Dissolve the above-mentioned DNA with 5 μl hybridizing buffer (5 X SSC, 0.2% SDS) and allow the DNA to be denatured for 5 min. Allow the sample to cool down to room temperature and be added dropwise onto the surface of DNA array. Cover the sample with glass slide and allow hybridization to last for 4-8 hr.

Testing for hybridizing signal:

Test the hybridizing signal of DNA chip with confocal fluorescent microscope or fluorescent scanner. Refer to FIG. 3 for the results.

### SAMPLE 2

Use the chip of this invention to test sickle cell anemia patients with HbS mutation. Take a blood sample of 1 ml from the patient to be tested and extract the genomic DNA of the patient with the commercialized genomic DNA extraction kit or the standard phenol extracting/ethanol depositing method. Amplify the exon, 1, 2, 3 of β globin gene with the design PCR (polymerase chain reaction) primers of three groups (5′ terminus of one primer modified with fluorescence). The amplified volume is 50 μl. Mix the PCR product and add 3 M pH 5.2 acidic acid buffer with the volume 1/10 time that of the sample. Add −20° C. for 30 min. Centrifuge at 1300 rpm for 10 min. and wash with 70% ethanol. Then allow to deposit and dry.

Hybridizing process on the surface of DNA chip:

Dissolve the above-mentioned DNA with 5 μl hybridizing buffer (5 X SSC, 0.2% SDS) and allow the DNA to be denatured for 5 min. Allow the sample to cool down to room temperature and be added dropwise onto the surface of DNA array. Cover the sample with glass slide and allow hybridization to last for 4-8 hr.

Testing for hybridizing signal:

Test the hybridizing signal of DNA chip with confocal fluorescent microscope or fluorescent scanner. Refer to FIG. 4 for the results.

The samples demonstrate the feasibility for the application of this invention on testing the mutation of hereditary anemia related genes. Hence, this invention is applicable to the early diagnosis and prenatal screening of hereditary anemia.

1. A kind of DNA chip for diagnosing the mutation of the hereditary anemia related genes, wherein fixed specific DNA probes for testing the above-said mutation on the glass slide, silica plate, membrane and macromolecular materials. The said probes are as follows:

| | |
|---|---|
| β (27-28) 1 | |
| TGG TGA GGC CCT GGG CAG | (SEQ ID NO:1) |
| β (27-28) 2 | |
| GGT GAG GCC CCT GGG CAG | (SEQ ID NO:2) |
| β (43) 1 | |
| GGT TCT TTG AGT CCT TT | (SEQ ID NO:3) |
| β (43) 2 | |
| GGT TCT TTT AGT CCT TT | (SEQ ID NO:4) |
| β (42 + T) 2 | |
| AGG TTC TTT TGA GTC CT | (SEQ ID NO:5) |
| IVS (2-1) 1 | |
| CTT CAG GGT GAG TCT | (SEQ ID NO:6) |
| IVS (2-1) 2 | |
| CTT CAG GAT GAG TCT | (SEQ ID NO:7) |
| β (1) 1 | |
| ACA GAC ACC ATG GTG CAC CT | (SEQ ID NO:8) |
| β (1) 2 | |
| ACA GAC ACC AGG GTG CAC CT | (SEQ ID NO:9) |
| β (8) 1 | |
| GAG GAG AAG TCT GCC | (SEQ ID NO:10) |
| β (8) 2 | |
| TGA GGA GGT CTG CCG | (SEQ ID NO:11) |
| β (8-9) 2 | |
| AGG AGA AGG TCT GCC | (SEQ ID NO:12) |
| β (37) 1 | |
| TAC CCT TGG ACC CAG | (SEQ ID NO:13) |
| β (37) 2 | |
| TAC CCT TAG ACC CAG | (SEQ ID NO:14) |
| P (+40-43) 1 | |
| GCA ACC TCA AAC AGA CA | (SEQ ID NO:15) |
| P (+40-43) 2 | |
| AGC AAC CTC AGA CAC CA | (SEQ ID NO:16) |
| P (1331, IVS1) 1 | |
| CAC CCT TAG GCT GCT GG | (SEQ ID NO:17) |
| P (IVS1) 2 | |
| CCC ACC CTG AGG CTG CT | (SEQ ID NO:18) |
| β (31) 2 | |
| CCC TTA GGT GCT GGT GG | (SEQ ID NO:19) |
| P (cap + 1) 1 | |
| ATT GCT TAC ATT TGC | (SEQ ID NO:20) |
| P (cap + 1) 2 | |
| ATT GCT TCC ATT TGC | (SEQ ID NO:21) |
| β (19) 1 | |
| AAG GTG AAC GTG GAT | (SEQ ID NO:22) |
| β (19) 2 | |
| AAG GTG AGC GTG GAT | (SEQ ID NO;23) |
| β (95 + A) 1 | |
| CTG TGA CAA GCT GCA | (SEQ ID NO:24) |
| β (95 + A) 2 | |
| TGT GAC AAA GCT GCA | (SEQ ID NO:25) |
| IVS (2-5) 1 | |
| AGG GTG AGT CTA TGG | (SEQ ID NO:26) |
| IVS (2-5) 2 | |
| AGG GTG ACT CTA TGG | (SEQ ID NO:27) |
| β (41-42) 1 | |
| CAG AGG TTC TTT GAG T | (SEQ ID NO:28) |
| β (41-42) 2 | |
| CAG AGG TTG AGT CCT T | (SEQ ID NO:29) |
| IVS (2-654) 1 | |
| GTT AAG GCA ATA GCA | (SEQ ID NO:30) |
| IVS (2-654) 2 | |
| GTT AAG GTA ATA GCA | (SEQ ID NO:31) |
| β (17) 1 | |
| CTG TGG GGC AAG GTG AAC | (SEQ ID NO:32) |
| β (17) 2 | |
| CTG TGG GGC TAG GTG AAC | (SEQ ID NO:33) |
| β (71-72) 1 | |
| TGC CTT TAG TGA TGG | (SEQ ID NO:34) |
| β (71-72) 2 | |
| TGC CTT TAA GTG ATG | (SEQ ID NO:35) |
| β (71-72) 3 | |
| TGC CTT TTA GTG ATG | (SEQ ID NO:36) |
| IVS (1-5) 1 | |
| CAG GTT GGT ATC AAG | (SEQ ID NO:37) |
| IVS (1-5) 2 | |
| CAG GTT GCT ATC AAG | (SEQ ID NO:38) |
| IVS (1-1) 1 | |
| TGG GCA GGT TGG TAT | (SEQ ID NO:39) |
| IVS (1-1) 2 | |
| TGG GCA GTT TGG TAT | (SEQ ID NO:40) |
| β (30) 2 | |
| CTG GGC GGG TTG GTA | (SEQ ID NO:41) |
| P (−28) 1 | |
| GGG CAT AAG AGT CAG | (SEQ ID NO:42) |
| P (−28) 2 | |
| GGG CAT AGG AGT CAG | (SEQ ID NO:43) |
| P (−29) 2 | |
| TGG GCA TGG AAG TCA | (SEQ ID NO:44) |
| P (−30) 1 | |
| CTG GGC ATA AAA GTC | (SEQ ID NO:45) |
| P (−30) 2 | |
| CTG GGC ACA AAA GTC | (SEQ ID NO:46) |
| P (−31) 2 | |
| GCT GGG CGT AAA AGT | (SEQ ID NO:47) |
| P (−32) 2 | |
| GGC TGG GAA TAA AAG | (SEQ ID NO:48) |
| β (14-15) 1 | |
| TAC TGC CCT GTG GGG CAA GG | (SEQ ID NO:49) |
| β (14-15) 2 | |
| TAC TGC CCT GGT GGG GCA AG | (SEQ ID NO:50) |
| HbE (26) 1 | |
| TGG TGG TGA GGC CCT | (SEQ ID NO:51) |
| HbE (26) 2 | |
| TGG TGG TAA GGC CCT | (SEQ ID NO:52) |

α-thalassemia, HbH, HbS and HbM hemoglobin abnormality.
Constant Spring Mutation

| | | |
|---|---|---|
| P (cs) 1 | ATA CCG TTA AGG TGG | (SEQ ID NO:53) |
| P (cs) 2 | ATA CCG TCA AGC TGG | (SEQ ID NO:54) |

Quong SZE Mutation

| | | |
|---|---|---|
| P (qs) 1 | GCC TCC CTG GAC AAG | (SEQ ID NO:55) |
| P (qs) 2 | GCC TCC CCG GAC AAG | (SEQ ID NO:56) |

HbS Sickle Cell Anemia Mutation

| | | |
|---|---|---|
| P (hbs) 1 | ACT CCT GAG GAG AAG | (SEQ ID NO:57) |
| P (hbs) 2 | ACT CCT GTG GAG AAG | (SEQ ID NO:58) |

Duan Mutation

| | | |
|---|---|---|
| P (duan) 1 | GTG GAC GAC ATG CCC | (SEQ ID NO:59) |
| P (duan) 2 | GTG GAC GCC ATG CCC | (SEQ ID NO:60) |

HbM Mutation

| | | |
|---|---|---|
| P (hbm) 1 | TAA GGG CCA CGG CAA | (SEQ ID NO:61) |
| P (hbm) 2 | TAA GGG CTA CGG CAA | (SEQ ID NO:62) |
| P (hbm) 3 | CGA CCT GCA CGC GCA | (SEQ ID NO:63) |
| P (hbm) 4 | CGA CCT GTA CGC GCA | (SEQ ID NO:64) |
| P (hbm) 5 | AAG AAA GTG CTC GGT | (SEQ ID NO:65) |
| P (hbm) 6 | AAG AAA GAG CTC GGT | (SEQ ID NO:66) |
| P (hbm) 7 | TGA GCT GCA CTG TGA | (SEQ ID NO:67) |
| P (hbm) 8 | TGA GCT GCA CTG TGA | (SEQ ID NO:68) |
| P (hbm) 9 | GAA GGC TCA TGG CAA | (SEQ ID NO:69) |
| P (hbm) 10 | GAA GGC TTA TGG CAA | (SEQ ID NO:70) |
